(19) United States
(12) Patent Application Publication
(10) Pub. No.: US 2019/0314143 A1
(43) Pub. Date: Oct. 17, 2019

(54) SURGICAL FIXATION SYSTEMS WITH INTEGRATED REINFORCEMENT CONSTRUCTS

(71) Applicant: ARTHREX, INC., Naples, FL (US)

(72) Inventors: Jeffrey Jackson, Holladay, UT (US); John Xerogeanes, Atlanta, GA (US); Jacob Jolly, Naples, FL (US)

(21) Appl. No.: 15/954,962

(22) Filed: Apr. 17, 2018

(51) Int. Cl. A61F 2/08; A61B 17/04

(52) U.S. Cl. CPC A61F 2/0811; A61B 17/0401; A61B 2017/00004; A61B 2017/0414; A61B 2017/0409; A61B 2017/0404

(57) ABSTRACT

This disclosure relates to surgical fixation systems and methods. The surgical fixation systems can include a fixation device and an adjustable loop connected to the fixation device. A reinforcement construct may be integrated with the adjustable loop.

### BACKGROUND

This disclosure relates to surgical fixation systems and methods for reinforcing a graft.

Tissue reconstruction surgeries, such as anterior cruciate ligament (ACL) reconstructions and posterior cruciate ligament (PCL) reconstructions, for example, typically involve drilling a tunnel through bone, positioning a substitute graft into the bone tunnel, and fixating the graft within the bone tunnel using a fixation device, such as a button, a screw, or the like. Some surgeons may desire to reinforce the graft prior to fixating the graft within the bone tunnel in order to increase its fixation strength.

### SUMMARY

This disclosure relates to surgical fixation systems and methods. The surgical fixation systems may include a fixation device and a loop connected to the fixation device. A graft may be carried by the loop. The graft may be reinforced by a reinforcement construct. The surgical fixation system can be used in various tissue reconstruction procedures, including but not limited to, ACL and PCL reconstructions.

A surgical fixation system according to an exemplary aspect of this disclosure may include, inter alia, a button, an adjustable loop connected to the button, and a reinforcement construct integrated with the adjustable loop. A reinforcement scaffold of the reinforcement construct may include a woven, rip-stop pattern that is attached to the adjustable loop. A suture loop of the reinforcement construct may include a continuous, uninterrupted suture loop that is attached to the reinforcement scaffold. A needle of the reinforcement construct may be attached to the continuous, uninterrupted suture loop.

A surgical fixation system according to another exemplary aspect of this disclosure may include, inter alia, a fixation device, a loop connected to the fixation device, and a reinforcement construct pre-assembled to the loop and including a reinforcement scaffold attached to the loop and a suture loop attached to the reinforcement scaffold.

A surgical method according to another exemplary aspect of this disclosure may include, inter alia, positioning a reinforcement scaffold of a reinforcement construct of a surgical fixation system against a first side of the graft. The reinforcement construct may be pre-assembled to an adjustable loop of the surgical fixation system. A graft may be positioned through an opening between suture strands of a suture loop of the reinforcement construct, and a needle of the reinforcement construct may be passed through the reinforcement scaffold and the graft to create a first stitched region. The graft may then be repositioned through the opening between the suture strands of the suture loop, and the needle may be repassed through the reinforcement scaffold and the graft to create a second stitched region that is distal to the first stitched region. The suture loop may then be passed through an opening of the adjustable loop, and the needle may be repassed again through the reinforcement scaffold and the graft to create a third stitched region that is between the first stitched region and the second stitched region.

### BRIEF DESCRIPTION OF THE DRAWINGS

FIG. 1 illustrates a surgical fixation system for performing a tissue reconstruction procedure according to an embodiment of this disclosure.

FIG. 2 illustrate an adjustable loop of the surgical fixation system of FIG. 1. The adjustable loop may include an integrated reinforcement construct.

FIGS. 3, 4, 5, 6, 7, 8, 9, 10, and 11 schematically illustrate a method for reinforcing a graft.

FIG. 12 schematically illustrates an exemplary use of a surgical fixation system that includes a reinforced graft as part of a tissue reconstruction procedure.

### DETAILED DESCRIPTION

This disclosure relates to surgical fixation systems. The surgical fixation systems may include a fixation device and a loop connected to the fixation device. A graft may be carried by the loop. The graft may be reinforced by a reinforcement construct. The surgical fixation system can be used in various tissue reconstruction procedures, including but not limited to, ACL and PCL reconstructions. Also disclosed are exemplary methods for reinforcing a graft of a surgical fixation system.

A surgical fixation system according to an exemplary aspect of this disclosure may include, inter alia, a button, an adjustable loop connected to the button, and a reinforcement construct integrated with the adjustable loop. A reinforcement scaffold of the reinforcement construct may include a woven, rip-stop pattern that is attached to the adjustable loop. A suture loop of the reinforcement construct may include a continuous, uninterrupted suture loop that is attached to the reinforcement scaffold. A needle of the reinforcement construct may be attached to the continuous, uninterrupted suture loop.

A surgical fixation system according to another exemplary aspect of this disclosure may include, inter alia, a fixation device, a loop connected to the fixation device, and a reinforcement construct pre-assembled to the loop and including a reinforcement scaffold attached to the loop and a suture loop attached to the reinforcement scaffold.

In a further embodiment, a fixation device of a surgical fixation system is a button.

In a further embodiment, a loop of a surgical fixation system is an adjustable loop that may include a first adjustable eyesplice loop received through a first aperture of a fixation device and a second adjustable eyesplice loop received through a second aperture of the fixation device.

In a further embodiment, an adjustable loop of a surgical fixation system may include a first free braid strand for adjusting a first adjustable eyesplice loop and a second free braid strand for adjusting a second adjustable eyesplice loop.

In a further embodiment, a first free braid strand of an adjustable loop of a surgical fixation system extends from a first spliced section of the adjustable loop and a second free braid strand extends from a second spliced section of the adjustable loop.

In a further embodiment, a reinforcement construct of a surgical fixation system may include a woven, rip-stop pattern.

In a further embodiment, a suture loop of a reinforcement construct of a surgical fixation system may include a continuous, uninterrupted suture loop.

In a further embodiment, a reinforcement construct of a surgical fixation system may include a needle.

In a further embodiment, a needle of a reinforcement construct of a surgical fixation system may be a free floating needle that is movable relative to a suture loop.

In a further embodiment, a needle of a reinforcement construct of a surgical fixation system may be swaged to the suture loop.

In a further embodiment, a reinforcement construct of a surgical fixation system may include a needle that is attached to a suture loop at an opposite end of the suture loop from a reinforcement scaffold.

A surgical method according to another exemplary aspect of this disclosure may include, inter alia, positioning a reinforcement scaffold of a reinforcement construct of a surgical fixation system against a first side of the graft. The reinforcement construct may be pre-assembled to an adjustable loop of the surgical fixation system. A graft may be positioned through an opening between suture strands of a suture loop of the reinforcement construct, and a needle of the reinforcement construct may be passed through the reinforcement scaffold and the graft to create a first stitched region. The graft may then be repositioned through the opening between the suture strands of the suture loop, and the needle may be repassed through the reinforcement scaffold and the graft to create a second stitched region that is distal to the first stitched region. The suture loop may then be passed through an opening of the adjustable loop, and the needle may be repassed again through the reinforcement scaffold and the graft to create a third stitched region that is between the first stitched region and the second stitched region.

In a further embodiment, a graft may be secured to a workstation prior to positioning a reinforcement scaffold of a surgical fixation system against the graft.

In a further embodiment, positioning a reinforcement scaffold of a reinforcement construct of a surgical fixation system against a graft may include aligning a distal aspect of the reinforcement scaffold with a distal aspect of the graft.

In a further embodiment, a surgical method may include passing a needle of a reinforcement construct of a surgical fixation system through a proximal portion of a graft before positioning the graft through an opening of a suture loop and prior to creating first and second stitched regions.

In a further embodiment, a surgical method may include clamping a reinforcement scaffold of a reinforcement construct of a surgical fixation system to a graft prior to positioning the graft through a suture loop opening and prior to creating first and second stitched regions.

In a further embodiment, a surgical method may include cutting off a needle from a suture loop of a surgical fixation system and tying suture strands of the suture loop into a knot.

In a further embodiment, a needle of a reinforcement construct of a surgical fixation system may be inserted in a direction from a first side to a second side of a graft each time the needle is passed through a reinforcement scaffold and the graft.

In a further embodiment, a reinforcement construct of a surgical fixation system may already be attached to an adjustable loop when the surgical fixation system is removed from its sterile packaging.

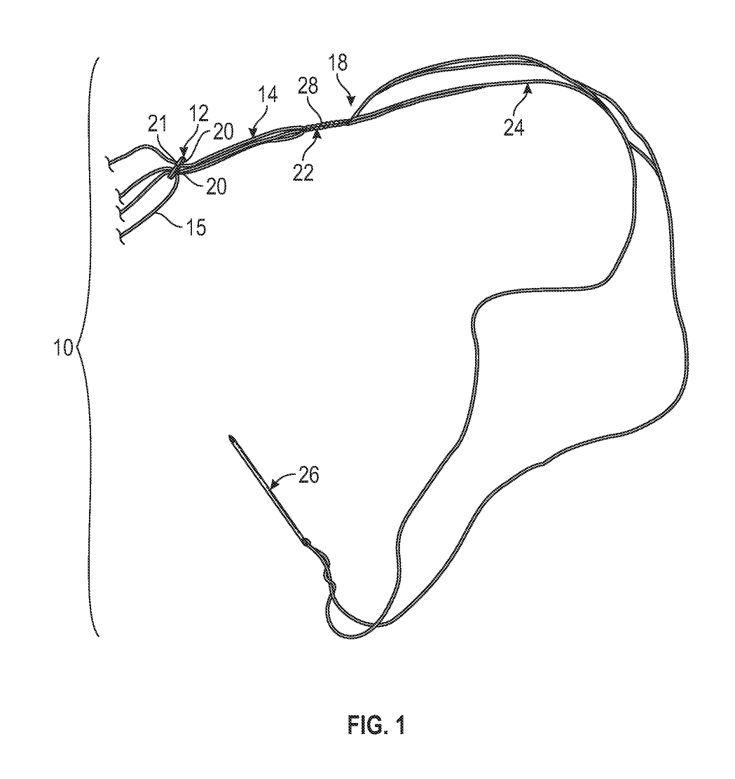

FIG. 1 illustrates an exemplary surgical fixation system 10. The surgical fixation system 10 may be used to perform a variety of tissue reconstruction procedures. The tissue reconstruction procedures could include any procedure that includes positioning a replacement graft within a bone tunnel to repair torn tissue. ACL and PCL reconstructions are two non-limiting examples of reconstruction procedures which could benefit from the surgical fixation systems 10 of this disclosure. The surgical fixation system 10 could additionally be used in procedures related to the ankle (e.g., syndesmosis procedures) and shoulder (e.g., acromioclavicular (AC) joint procedures). The surgical fixation system 10 could additionally be used in any tendon to bone fixation procedure (e.g., biceps repairs).

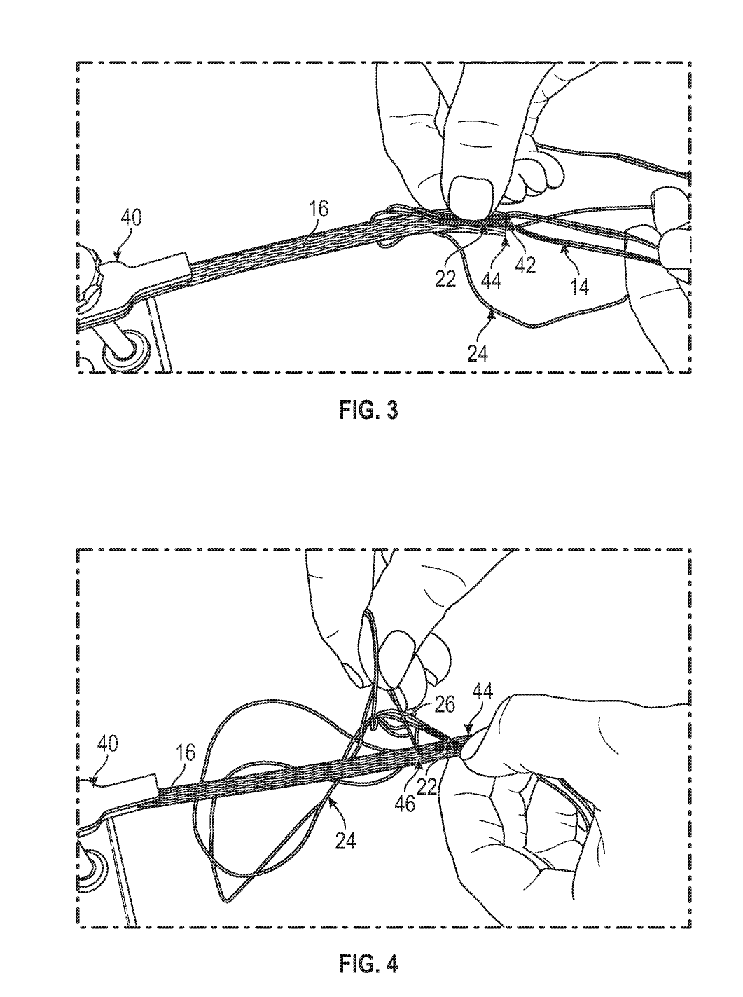

The surgical fixation system 10 may include, in this example, a fixation device 12, a loop 14, and a reinforcement construct 18. The loop 14 is connected to the fixation device 12. In an embodiment, the loop 14 carries a graft (see feature 16 of FIGS. 3-11) for fixating the graft relative to bone. In another embodiment, the loop 14 carries a filament (e.g., any thread-like material such as suture, etc.) for fixating the filament relative to bone.

The fixation device 12 may provide cortical bone fixation of a graft (or filament), for example, after the graft has been positioned within a bone tunnel. In an embodiment, the fixation device 12 is a button. However, fixation devices having other similar configurations could also be used. The fixation device 12 may be oblong or round and may be made of either metallic or polymeric materials within the scope of this disclosure.

In another embodiment, the fixation device 12 includes one or more apertures 20 formed through the body of the fixation device 12 for receiving the loop 14. The fixation device 12 of the embodiment of FIG. 1, for example, includes two apertures 20 for connecting the loop 14 to the fixation device 12. However, the fixation device 12 could include additional apertures or openings in excess of two within the scope of this disclosure.

The apertures 20 may be configured and arranged to receive the loop 14 of the surgical fixation system 10. A bridge 21 of the fixation device 12 may separate the apertures 20 from one another and provides a surface for carrying the loop 14 of the surgical fixation system 10. One of apertures 20 may additionally carry one or more filaments 15 for passing the fixation device 12 through a bone tunnel and/or for flipping the fixation device 12 relative to bone after exiting from the bone tunnel.

The reinforcement construct 18 may be integrated with a portion of the loop 14. In an embodiment, the reinforcement construct 18 is integrated with a portion of the loop 14 that is located at an opposite end from the portion of the loop 14 that rests over the bridge 21 of the fixation device 12.

In an embodiment, the reinforcement construct 18 is pre-assembled to the loop 14. In this disclosure, the term “pre-assembled” is intended to denote that the reinforcement construct 18 is affixed to the loop 14 during the manufacturing process rather than during or just prior to the surgical procedure. Thereby, surgeons or other medical personnel are not required to physically stitch or otherwise attach the reinforcement construct 18 to the loop 14 upon removing the surgical fixation system 10 from its sterile packaging.

As discussed in greater detail below, the reinforcement construct 18 may be used to attach a graft to the surgical fixation system 10 and to reinforce the graft prior to fixating the graft within a bone tunnel by implanting the surgical fixation system 10. The reinforcement construct 18 may include a reinforcement scaffold 22, a suture loop 24, and a needle 26.

The reinforcement scaffold 22 may be attached to the loop 14 of the surgical fixation system 10 by swaging, sewing, knotting, stitching, splicing, weaving, gluing, or by any other fixation technique. In an embodiment, the reinforcement scaffold 22 is looped around the loop 14 and then sewed to itself to attach the reinforcement scaffold to the loop 14. The reinforcement scaffold 22 may include any reinforcement material that can be attached to a graft (e.g., tendon graft, ligament graft, synthetic graft, etc.). The reinforcement scaffold 22 may include suture, tape, weave, or mesh, or any combinations of these materials. In an embodiment, the reinforcement scaffold 22 includes a woven, rip-stop pattern 28. The woven, rip-stop pattern 28 may be formed, for example, by interweaving threads at regular intervals in a crosshatch-like pattern.

The reinforcement scaffold 22 may also be bioabsorbable for resorbing away after a period of time in the body, or may be provided with various medicinal or therapeutic agents such as antiseptics, antibiotics, drugs, pharmaceutical agents, hormones, and/or growth materials (e.g., autogenous growth factors such as platelet-rich plasma (PRP), autologous factors, autologous-conditioned plasma (ACP), etc.). The reinforcement scaffold 22 may include any size, shape, and geometry for reinforcing the graft that is to be sutured. In an embodiment, the reinforcement scaffold 22 includes a rectangular configuration or any other configuration that provides a surface area upon which suturing/stitching may be conducted.

The suture loop 24 may be attached to the reinforcement scaffold 22 by swaging, knotting, or by any other fixation technique. In an embodiment, the suture loop 24 is a continuous suture loop (i.e., the suture loop 24 may lack free ends). The suture loop 24 may be constructed of any flexible, thread-like material or suture material. In an embodiment, the suture loop 24 is made of FiberWire® suture, which is available for purchase from Arthrex, Inc. of Naples, Fla.

The suture loop 24 may be attached to the needle 26 at an opposite end of the suture loop 24 from the reinforcement scaffold 22. In an embodiment, the suture loop 24 is swaged onto the needle 26. In another embodiment, the needle 26 is a free floating needle that may move freely on the suture loop 24 for re-centering itself after passing through graft/tissue and to facilitate even tensioning. In yet another embodiment, the needle 26 includes a relatively thin and straight configuration that is simple to handle.

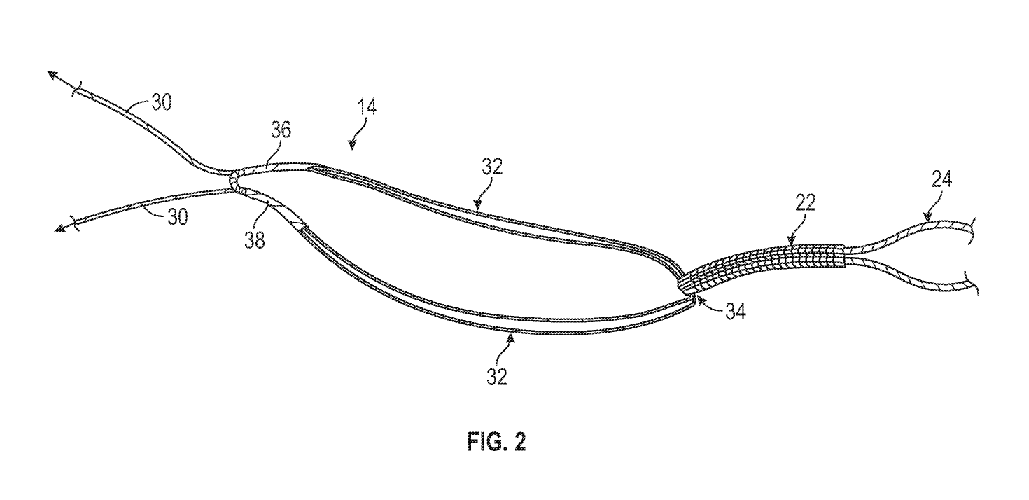

FIG. 2 illustrates additional features of the loop 14 of the surgical fixation system 10 of FIG. 1. The fixation device 12 is removed in FIG. 2 to better illustrate the configuration of the loop 14.

In an embodiment, the loop 14 is an adjustable loop made of a flexible material, and in this example, may include an adjustable length and/or perimeter. Free braid strands 30 of the loop 14, which may also be referred to as shortening strands, may be pulled to reduce the size of the loop 14. For example, the loop 14 may be adjusted in a first direction by pulling the free braid stands 30 but is prevented from loosening in the opposite direction due to applied internal tensile forces. In an embodiment, the free braid strands 30 extend from spliced sections 36, 38 of the loop 14. The spliced sections 36, 38 may act as locking mechanisms for preventing the loosening of the free braid strands 30.

The loop 14 may additionally include one or more adjustable eyesplice loops 32, which may be formed by splicing the flexible material that is used to form the loop 14 through itself at each of the spliced sections 36, 38. The loop 14 may be connected to the fixation device 12 prior to completely forming the loop 14. The free braid strands 30 may be pulled to constrict the size of the adjustable eyesplice loops 32 and thus may change the overall size of the loop 14.

The adjustable eyesplice loops 32 may be interlinked at an interconnection 34. In an embodiment, the reinforcement scaffold 22 is affixed to the loop 14 at the interconnection 34.

FIGS. 3-11, with continued reference to FIGS. 1-2, schematically illustrate an exemplary method for securing the reinforcement construct 18 of the surgical fixation system 10 to a graft 16 in order to reinforce the graft 16. FIGS. 3 through 11 illustrate, in sequential order, an exemplary embodiment for reinforcing the graft 16 by employing the reinforcement construct 18 of the surgical fixation system 10. However, fewer or additional steps than are recited below could be performed within the scope of this disclosure, and the recited order of steps is not intended to limit this disclosure.

The graft 16 could include tissue, tendon, ligament, filament (e.g., suture), synthetic material, biologic material, bone, or any combinations of such materials. In an embodiment, the graft 16 is a single-ended graft that only includes one end that is suitable for attachment to the surgical fixation system 10. However, the graft 16 could include any type of graft configuration within the scope of this disclosure.

Referring first to FIG. 3, the graft 16 may be secured relative to a graft workstation 40. The reinforcement scaffold 22 may then be laid on top of the portion of the graft 16 that is to be stitched to the surgical fixation system 10. In an embodiment, the reinforcement scaffold 22 is positioned over the graft 16 such that a distal aspect 42 of the reinforcement scaffold 22 is aligned with a distal aspect 44 of the graft 16. The distal aspect 44 of the graft 16 includes the end of the graft 16 that is suitable for attachment to the surgical fixation system 10.

Next, as shown in FIG. 4, the needle 26 of the reinforcement construct 18 is passed through a proximal portion 46 of the graft 16. This first pass of the needle 26 through the graft 16 begins the stitching process for anchoring the reinforcement scaffold 22 to the graft 16. In an embodiment, the proximal portion 46 is proximal of the distal aspect 44 of the graft 16.

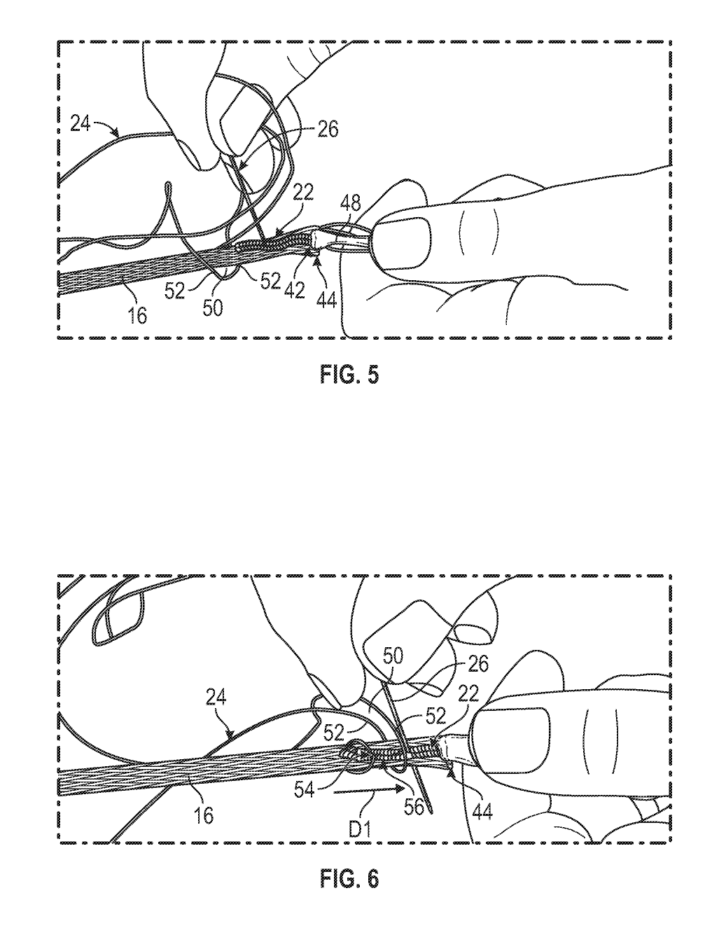

FIG. 5 illustrates additional steps of the stitching process for anchoring the reinforcement scaffold 22 to the graft 16. The distal aspect 42 of the reinforcement scaffold 22 may be held in place relative to the distal aspect 44 of the graft 16 by a clamp 48 (e.g., an Alice clamp) in order to hold the graft 16 taut during the stitching process.

In an embodiment, the stitching process is a whipstitching process. During an exemplary whipstitching process, a plurality of stitching passes are made through both the reinforcement scaffold 22 and the graft 16 in order to stitch the reinforcement scaffold 22 to the graft 16 using the suture loop 24 and the needle 26 of the reinforcement construct 18. For example, after positioning the suture loop 24 around the graft 16 such that the graft 16 extends through an opening 50 between suture strands 52 of the suture loop 24, the needle 26 is passed through both the reinforcement scaffold 22 and the graft 16 to form a first stitched region 54 (see FIG. 6).

Referring now to FIG. 6, after passing and tensioning the first stitched region 54, the suture strands 52 of the suture loop 24 are again spread on either side of the graft 16, thereby repositioning the graft 16 through the opening 50 between the suture strands 52. The needle 26 may then be passed again through both the reinforcement scaffold 22 and the graft 16 at a location that is distal to the first stitched region 54 (i.e., toward the distal aspect 44 of the graft 16) to form a second stitched region 56. This process may be repeated in a direction D1 toward the distal aspect 44 of the graft 16 until a desired number of stitched regions have been formed (in the illustrated embodiment, three stitched regions 54, 56, 57 are formed). In an embodiment, the needle 26 is inserted on the same side of the graft during each stitching pass of the suture loop 24 through the reinforcement scaffold 22 and the graft 16.

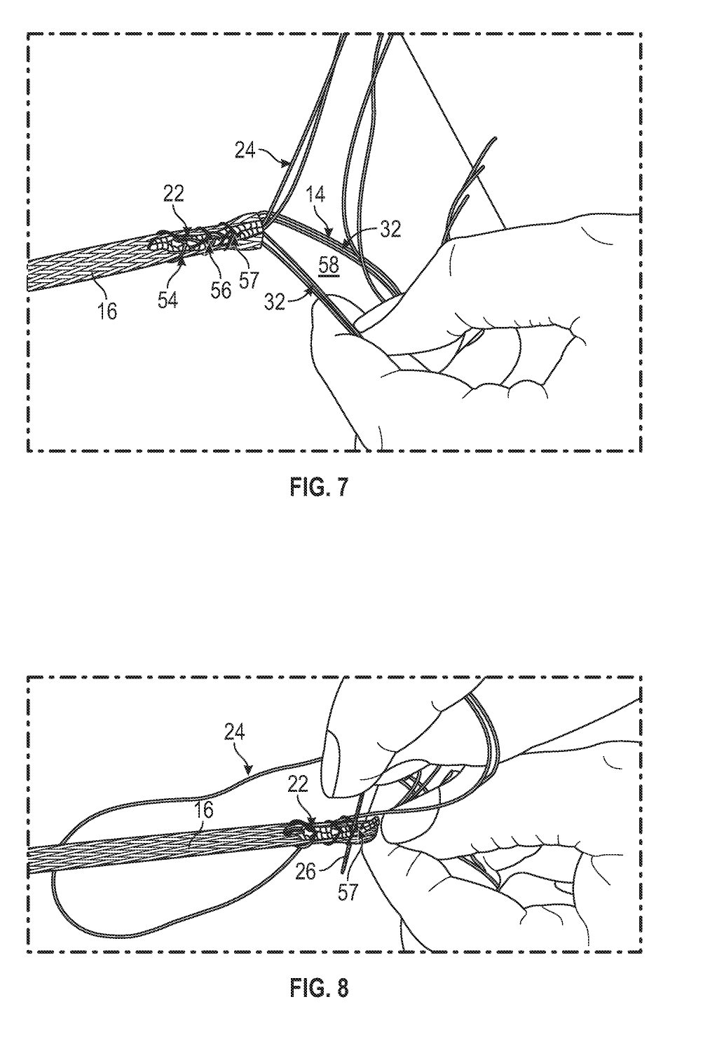

After a desired number of stitched regions have been formed, the suture loop 24 may be passed through an opening 58 of the suture loop 14. The opening 58 may be located between the adjustable eyesplice loops 32, for example. This step is shown in FIG. 7. Subsequently, as shown in FIG. 8, the needle 26 may be passed through the reinforcement scaffold 22 and the graft 16 at a location that is just proximal to the distal-most stitched region (in this example, the stitched region 57) in order to lock the stitching into the graft 16.

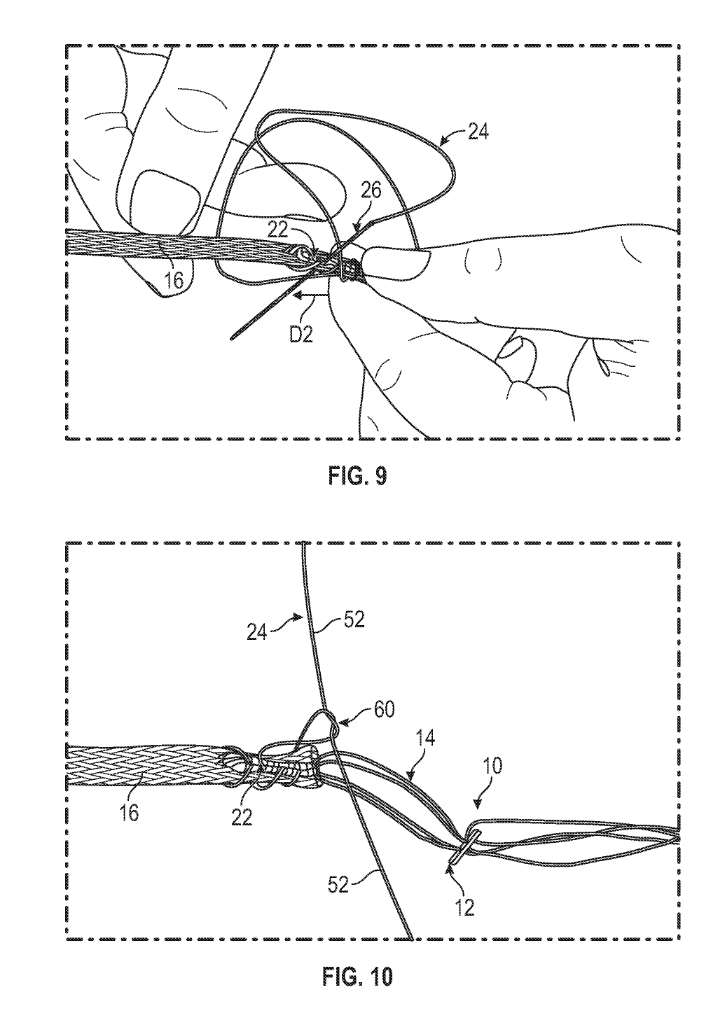

Referring to FIG. 9, the whipstitching technique described above may next be continued in a second direction D2 that is opposite from the first direction D1 in order to create additional stitched regions in the reinforcement scaffold 22 and the graft 16. In an embodiment, these additional stitched regions are placed between the stitched regions 54, 56, and 57 formed during the steps shown in FIGS. 5-6.

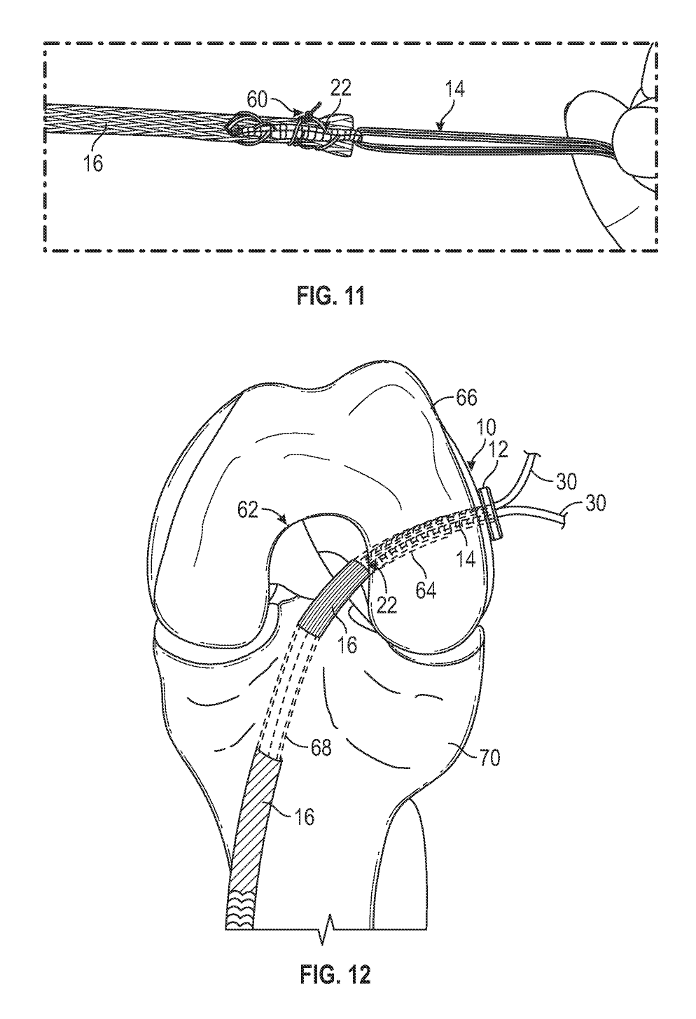

Once a desired number of additional stitched regions have been formed, the needle 26 may be cut off of the suture loop 24 and the now separated suture strands 52 of the suture loop 24 may then be tied into a knot 60 to complete the stitching process. These final steps are illustrated in FIGS. 10 and 11. The graft 16 is fixedly secured to the surgical fixation system 10 once the knot 60 is tied.

FIG. 12 illustrates an exemplary surgical use of the surgical fixation system 10 of FIGS. 1-11 during a tissue reconstruction procedure, such as an ACL reconstruction procedure. However, this disclosure is not limited to ACL reconstruction procedures, and the surgical fixation system 10 could be used in a variety of reconstruction procedures within the scope of this disclosure.

The surgical fixation system 10, including the fixedly secured graft 16, may be implanted within a joint 62 (e.g., a knee joint) to repair a torn tissue (e.g., a torn ACL). Prior to positioning the surgical fixation system 10 within the joint 62, a first bone tunnel 64 (e.g., a socket) may be formed in a first bone 66 (e.g., a femur) and a second bone tunnel 68 (e.g., a passage) may be formed in a second bone 70 (e.g., a tibia). The first bone tunnel 64 and the second bone tunnel 68 may be formed using known drilling techniques to establish voids within the first and second bones 66, 70 for accommodating the surgical fixation system 10.

In an exemplary embodiment, the surgical fixation system 10 is implanted by passing the fixation device 12 through the first bone tunnel 64 and the second bone tunnel 68. The fixation device 12 may be pulled through the first and second bone tunnels 64, 68 using a passing suture (not shown) and may self-flip onto the cortex of the first bone 66 once tension is released on the passing suture.

After passing and flipping the fixation device 12, the loop 14 and the integrally fixated graft 16 are positioned within the first bone tunnel 64. The free braid strands 30 may be pulled to adjust the size of the loop 14 and to aid the positioning of the loop 14 and the graft 16 within the first bone tunnel 64. The loop 14 may suspend the graft 16 within portions of the first bone tunnel 64 and the second bone tunnel 68.

Fixation of the graft 16 relative to the second bone 70 can be achieved in a variety of ways. For example, the graft 16 may be fixated within the second bone tunnel 68 using an interference screw, a suture anchor, or an additional surgical fixation system that includes a second fixation device and a second loop.

The surgical fixation systems of this disclosure simplify and reduce surgical steps by integrating reinforcement constructs into loops of the surgical fixation systems. The reinforcement constructs reinforce the grafts, thereby increasing the strength at the loop/graft interface and reducing the likelihood that the loop becomes displaced away from the graft.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A surgical fixation system, comprising:
a button;
an adjustable loop connected to the button; and
a reinforcement construct integrated with the adjustable loop;
wherein a reinforcement scaffold of the reinforcement construct includes a woven, rip-stop pattern that is attached to the adjustable loop;
wherein a suture loop of the reinforcement construct includes a continuous, uninterrupted suture loop that is attached to the reinforcement scaffold;
wherein a needle of the reinforcement construct is attached to the continuous, uninterrupted suture loop.

2. A surgical fixation system, comprising:
a fixation device;
a loop connected to the fixation device; and
a reinforcement construct pre-assembled to the loop and including a reinforcement scaffold attached to the loop and a suture loop attached to the reinforcement scaffold.

3. The system as recited in claim 2, wherein the fixation device is a button.

4. The system as recited in claim 2, wherein the loop is an adjustable loop that includes a first adjustable eyesplice loop received through a first aperture of the fixation device and a second adjustable eyesplice loop received through a second aperture of the fixation device.

5. The system as recited in claim 4, wherein the adjustable loop includes a first free braid strand for adjusting the first adjustable eyesplice loop and a second free braid strand for adjusting the second adjustable eyesplice loop.

6. The system as recited in claim 5, wherein the first free braid strand extends from a first spliced section of the adjustable loop and the second free braid strand extends from a second spliced section of the adjustable loop.

7. The system as recited in claim 2, wherein the reinforcement construct includes a woven, rip-stop pattern.

8. The system as recited in claim 2, wherein the suture loop of the reinforcement construct includes a continuous, uninterrupted suture loop.

9. The system as recited in claim 2, wherein the reinforcement construct includes a needle.

10. The system as recited in claim 9, wherein the needle is a free floating needle that is movable relative to the suture loop.

11. The system as recited in claim 9, wherein the needle is swaged to the suture loop.

12. The system as recited in claim 9, wherein the reinforcement construct includes a needle that is attached to the suture loop at an opposite end of the suture loop from the reinforcement scaffold.

13. A method for reinforcing a graft, comprising:
positioning a reinforcement scaffold of a reinforcement construct of a surgical fixation system against a first side of the graft, wherein the reinforcement construct is pre-assembled to an adjustable loop of the surgical fixation system;
positioning the graft through an opening between suture strands of a suture loop of the reinforcement construct;
passing a needle of the reinforcement construct through the reinforcement scaffold and the graft to create a first stitched region;
repositioning the graft through the opening between the suture strands of the suture loop;
repassing the needle through the reinforcement scaffold and the graft to create a second stitched region that is distal to the first stitched region;
passing the suture loop through an opening of the adjustable loop; and
repassing the needle through the reinforcement scaffold and the graft to create a third stitched region that is between the first stitched region and the second stitched region.

14. The method as recited in claim 13, comprising:
securing the graft to a graft workstation prior to positioning the reinforcement scaffold.

15. The method as recited in claim 13, wherein positioning the reinforcement scaffold includes:
aligning a distal aspect of the reinforcement scaffold with a distal aspect of the graft.

16. The method as recited in claim 13, comprising:
passing the needle through a proximal portion of the graft before positioning the graft through the opening and creating the first and second stitched regions.

17. The method as recited in claim 13, comprising:
clamping the reinforcement scaffold to the graft prior to positioning the graft through the opening and creating the first and second stitched regions.

18. The method as recited in claim 13, comprising:
cutting off the needle from the suture loop; and
tying the suture strands of the suture loop into a knot.

19. The method as recited in claim 13, wherein the needle is inserted in a direction from the first side to a second side of the graft each time the needle is passed through the reinforcement scaffold and the graft.

20. The method as recited in claim 13, wherein the reinforcement construct is already attached to the adjustable loop when the surgical fixation system is removed from its sterile packaging.
